(19) United States
(12) Patent Application Publication
(10) Pub. No.: US 2013/0310927 A1
(43) Pub. Date: Nov. 21, 2013

(54) Implantable Valve System

(71) Applicant: James Quintessenza, Madeira Beach, FL (US)

(72) Inventor: James Quintessenza, Madeira Beach, FL (US)

(21) Appl. No.: 13/475,938

(22) Filed: May 18, 2012

(51) Int. Cl. A61F 2/24

(52) U.S. Cl. 623/2.12

(57) ABSTRACT

An implantable valve system comprises a flexible tube and a flexible valve structure. The flexible tube and flexible valve structure can be made of a synthetic polymer material. The flexible valve structure is disposed within the flexible tube. The flexible valve structure can comprises a first leaflet, a second leaflet, and a valve orifice between the first leaflet and the second leaflet. Additional leaflets are possible. Each of the first leaflet and the second leaflet comprises an upper portion forming the valve orifice and a lower portion attached to the flexible tube. The valve orifice has a maximum circumference less than an internal circumference of the flexible tube.

### FIELD OF THE INVENTION

This invention relates generally to implantable medical devices and, more particularly, an implantable valve system.

### BACKGROUND OF THE INVENTION

Implantable valves are used to treat many medical conditions. Valves are implanted in blood vessels and other parts of the anatomy. Valves are implanted to replace or supplement an existing valve that is diseased, defective, and/or malfunctioning. Valves could also be implanted in a part of the anatomy where valvular function was not present before.

Once in the patient, implanted valves are exposed to pulling forces and fluid pressures which can lead to damage and/or suboptimal valvular function. For example, the implanted valve can tear or rupture due to extreme physical exertion by the patient.

The implanted valve must also reliably open and close. If sized improperly in relation to its surroundings, moving elements of the valve orifice may not function reliably. For example, the moving elements may become stuck in an open configuration or may fail to provide a sufficient fluid seal.

Accordingly, there is a need for an implantable valve system that resists tearing and rupturing after implantation and which reliably maintains proper valvular function.

### SUMMARY OF THE INVENTION

Briefly and in general terms, the present invention is directed to an implantable valve system.

In aspect of the present invention, a system comprises a flexible tube, and a flexible valve structure within the flexible tube. The flexible valve structure comprises a first leaflet, a second leaflet, and a valve orifice between the first leaflet and the second leaflet. Each of the first leaflet and the second leaflet comprises an upper portion forming the valve orifice and a lower portion attached to the flexible tube. The valve orifice has a maximum circumference less than an internal circumference of the flexible tube.

In some aspects, each of the lower portions comprises a curved attachment region attached to the flexible tube.

In some aspects, the curved attachment region corresponds to a substantially parabolic edge of the lower portion.

In some aspects, the system further comprises sutures connecting the flexible tube to the curved attachment region of each lower portion.

In some aspects, each of the upper portions includes a top edge sized at about half of the internal circumference of the flexible tube.

In some aspects, the upper portion of the first leaflet is joined to the upper portion of the second leaflet at a first commissural region and at a second commissural region, and the valve orifice is bounded by the first commissural region and the second commissural region.

In some aspects, the system further comprises a first attachment suture at the first commissural region and a second attachment suture at the second commissural region. The first attachment suture connects the first commissural region to the flexible tube. The second attachment suture connects the second commissural region to the flexible tube.

In some aspects, the system further comprises a first commissural suture at the first commissural region and a second commissural suture at the second commissural region. The first commissural suture is located at or about an end of the valve orifice, and the second commissural suture is located at or about another end of the valve orifice.

In some aspects, each of the upper portions includes a top edge. Each top edge comprises end segments and a medial segment connecting the end segments. The first and second commissural regions join the end segments of the first leaflet to the end segments of the second leaflet. The medial segments define the valve orifice and are configured to move relative to each other from a separated state to a coaptated state.

In some aspects, each of the end segments is sized from about 1 mm to about 2 mm.

In some aspects, the top edge is sized at about half of the internal circumference of the flexible tube.

In some aspects, the flexible tube is configured to maintain an internal circumference without substantial enlargement upon application of an expansion force to the flexible tube.

In some aspects, the flexible tube is configured to stretch axially.

In some aspects, the first leaflet and the second leaflet are formed of a single, continuous sheet material that is neither human tissue nor animal tissue.

In some aspects, the flexible tube is formed of a material that is neither human tissue nor animal tissue.

In some aspects, the flexible tube, the first leaflet, and the second leaflet are formed of PTFE.

In some aspects, the flexible tube has an inner surface having a texture that allows for ingrowth or adhesion of biological tissue to the inner surface when implanted in a human or animal body.

In some aspects, the flexible tube has an outer surface that that is substantially fluid- tight.

In some aspects, the flexible tube has an outer surface that is substantially smooth and resistant to tissue ingrowth in comparison to the inner surface.

In some aspects, the system further comprises a tubular stent. The flexible tube is disposed within the tubular stent. The tubular stent comprises interconnected stent struts.

In some aspects, the system further comprises a catheter carrying the flexible tube and the flexible valve. The catheter extends through the flexible tube and the valve orifice.

The features and advantages of the invention will be more readily understood from the following detailed description which should be read in conjunction with the accompanying drawings.

### BRIEF DESCRIPTION OF THE DRAWINGS

FIG. 1 is a perspective view of an exemplary valve system, showing a flexible tube containing a flexible valve structure.

FIG. 2 is a perspective view of the flexible valve structure, showing an open valve orifice.

FIG. 3 is a perspective view of the flexible valve structure, showing a closed valve orifice.

FIG. 4 is an enlarged perspective view of the flexible valve structure of FIG. 2.

FIG. 5 is an enlarged perspective view of the flexible valve structure of FIG. 3.

FIG. 6 is a perspective view of an exemplary valve system, showing a tubular stent carrying a flexible tube and a flexible valve structure.

FIG. 7 is a perspective view of an exemplary valve system, showing a catheter carrying a tubular stent, a flexible tube, and a flexible valve structure.

FIG. 8 is a flow diagram showing a method of making a valve system.

### DETAILED DESCRIPTION OF EXEMPLARY EMBODIMENTS

As used herein, any term of approximation such as, without limitation, “near”, “about”, “approximately”, “substantially”, “essentially” and the like mean that the word or phrase modified by the term of approximation need not be exactly that which is written but may vary from that written description to some extent. The extent to which the description may vary will depend on how great a change can be instituted and have a person of ordinary skill in the art recognize the modified version as still having the properties, characteristics and capabilities of the modified word or phrase. For example and without limitation, a feature that is described as “substantially equal” to a second feature encompasses the features being exactly equal and the features being readily recognized by a person of ordinary skilled in the art as being equal although the features are not exactly the same.

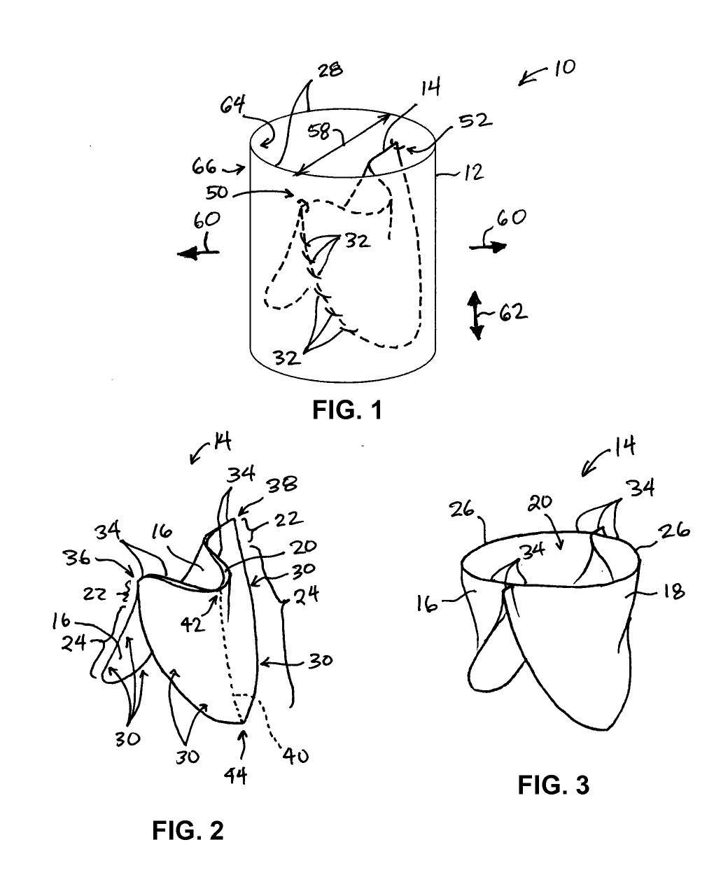

Referring now in more detail to the exemplary drawings for purposes of illustrating exemplary embodiments of the invention, wherein like reference numerals designate corresponding or like elements among the several views, there is shown in FIG. 1 an exemplary valve system 10.

In some embodiments, valve system 10 is an implantable prosthesis, which is a device that is totally or partly introduced, surgically or medically, into a patient's body (animal and human). The duration of implantation may be essentially permanent, i.e., intended to remain in place for the remaining lifespan of the patient; until the device biodegrades; or until the device is physically removed.

Valve system 10 includes flexible tube 12 and flexible valve structure 14. Flexible valve structure 14 is secured within flexible tube 12. FIGS. 2 and 3 show flexible valve structure 14 without flexible tube 12 for ease of illustration and for clarity.

As shown in FIG. 2, flexible valve structure 14 comprises first leaflet 16, second leaflet 18, and valve orifice 20. Valve orifice 20 is configured to open (FIG. 3) and close (FIG. 2) in response to fluid pressure and is located between first leaflet 16 and the second leaflet 20.

Each of first leaflet 16 and the second leaflet 20 comprises upper portion 22 and lower portion 24. Upper portions 22 form valve orifice 20. Lower portions 24 are attached to flexible tube 12.

In the illustrated exemplary embodiments, flexible valve structure 14 has two leaflets. In some embodiments, flexible valve structure 14 includes three or more leaflets.

Referring to FIG. 3, valve orifice 20 has a maximum circumference less than an internal circumference of flexible tube 14. The maximum circumference of valve orifice 20 can be measured along peripheral edge 26. The internal circumference of flexible tube 14 can be measured along peripheral edge 28 (FIG. 1).

Peripheral edges 26, 28 need not be perfectly circular, and the term “circumference” is not limited to the dimension of a perfect circle. Due to flexibility of flexible tube 12 and flexible valve structure 14, peripheral edge 26, 28 can have an irregular shape before, during, and after implantation in a patient. With peripheral edges 26, 28 having any of a circular shape, non-circular shape, and irregular shape, the circumferences refer to the total distance measured along and completely around the peripheral edges.

Each lower portion 24 comprises curved attachment region 30 which are attached to flexible tube 12 (not illustrated in FIGS. 2 and 3).

In some embodiments, curved attachment region 30 corresponds to a substantially parabolic edge of lower portion 24.

In some embodiments, curved attachment region 30 has the shape of a bishop miter. Curved attachment region 30 corresponds to a substantially parabolic parabolic edge with a sharp point 44 at the center of the leaflet. The sharp point 44 forms the apex of lower portion 24. Applicant has found that the substantially parabolic edge with sharp point 44 provides good valvular function.

In some embodiments, curved attachment region 30 corresponds to substantially parabolic shapes other than the exemplary illustrations herein.

In some embodiments, sutures 32 (FIG. 1) connect flexible tube 12 to curved attachment region 30 of each lower portion. Only a few sutures are illustrated for ease of illustration. It will be understood that additional sutures are used so that the entire length of attachment region 30 (for each of the leaflets) is secured to flexible tube 12.

Referring to FIG. 2 upper portion 22 of each leaflet 16, 18 includes top edge 34. Top edges 34 are sized equally.

In some embodiments, each top edge 34 sized at about half of the internal circumference of flexible tube 12. The size of top edge 34 is the length measured from point 36 to point 38. For example, each of top edge 34 can be from 0.4 to 0.7 of the internal circumference of flexible tube 12. As indicated above, the internal circumference of flexible tube 14 can be measured along peripheral edge 28 (FIG. 1).

Top edge 34 being sized at about half of the internal circumference of flexible tube 12 has been found by applicant to provide good valvular function. Valvular function includes the ability of valve orifice 20 to open and seal in response to fluid pressure.

It is possible to use other sizes for top edge 34 in relation to the internal circumference of flexible tube 12.

In some embodiments, axial dimension 40 of each leaflet 16, 18 is less than the size of top edge 34.

In some embodiments, axial dimension 40 of each leaflet 16, 18 is about 10% less than the size of top edge 34. Axial dimension 40 can be measured from the midpoint 42 of top edge 34 and to apex 44 of lower portion 18. Axial dimension 40 corresponds to the maximum height of each leaflet 16, 18. The size of top edge 34 corresponds to the maximum width of each leaflet 16, 18.

Axial dimension 40 being about 10% less than the size of top edge 34 has been found by applicant to provide good valvular function. For example, axial dimension 40 can be 7% to 13% less than the size of top edge 34

It is possible to use other sizes for axial dimension 40 in relation to top edge 34.

Referring to FIG. 3, portions of top edge 34 of first leaflet 16 is fixedly joined to portions of top edge 34 of second leaflet 16. Another portion of top edge 34 of first leaflet 16 is located opposite another portion of top edge 34 of second leaflet 16, and these top edge portions are not fixedly connected to each other and thereby form valve orifice 20.

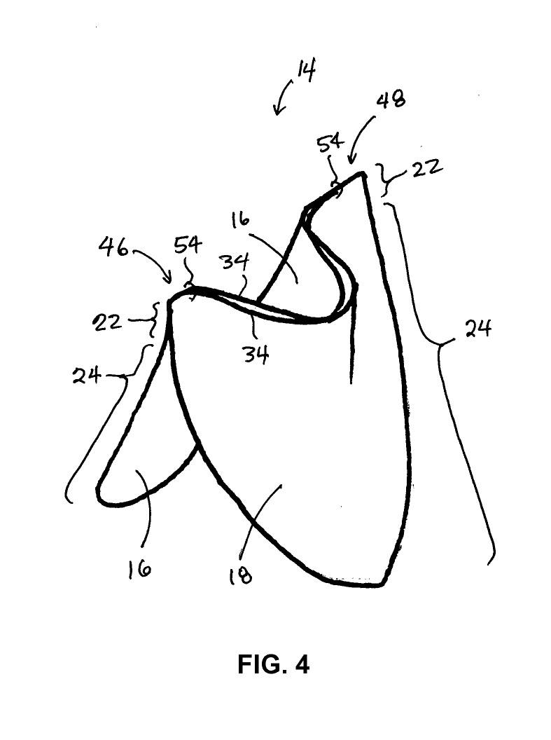

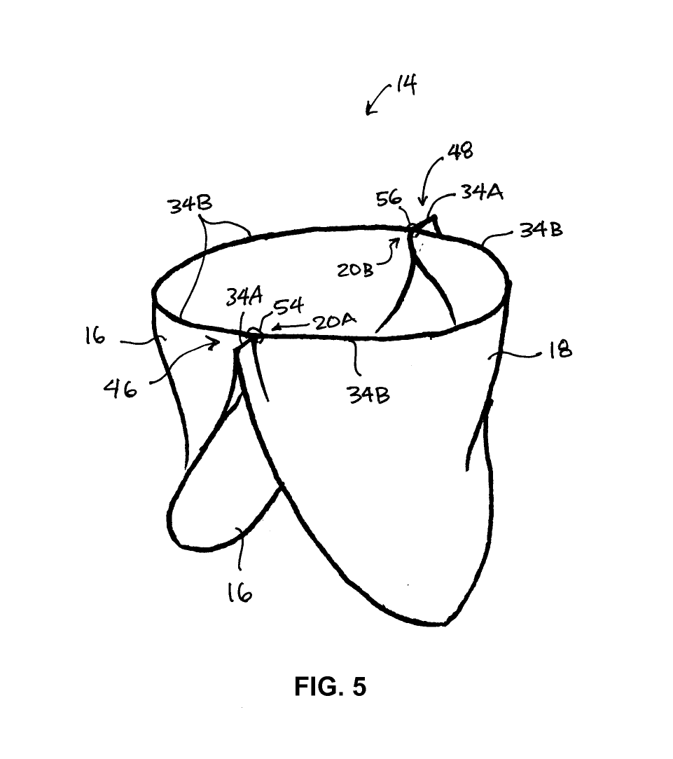

Referring to the enlarged views of FIGS. 4 and 5, upper portion 22 of first leaflet 16 is joined to upper portion 22 of second leaflet 18 at first commissural region 46 and at second commissural region 48. Valve orifice 20 is bounded by first commissural region 46 and second commissural region 48.

In some embodiments, first attachment suture 50 (FIG. 1) is located at first commissural region 46, and second attachment suture 52 (FIG. 1) at second commissural region 48. First attachment suture 50 connects first commissural region 46 to flexible tube 12. Second attachment suture 52 connects second commissural region 48 to flexible tube 12.

In some embodiments, first attachment suture 50 can be located at or about point 36 (FIG. 2) on top edges 34, and second attachment suture 52 can be located at or about point 38 (FIG. 2) on top edges 34.

Referring to FIGS. 4 and 5, in some embodiments first commissural suture 54 is located at first commissural region 46, and second commissural suture 56 is located at second commissural region. Referring to FIG. 5, first commissural suture 54 is at or about an end 20A of the valve orifice 20, and second commissural suture 56 located at or about another end 20B of valve orifice 20. Valve orifice ends 20A, 20B are defined as points where leaflets 16, 28 begin to separate from each other.

As indicated above, each leaflet 16, 18 includes top edge 34. Each top edge 34 (FIG. 3) comprises end segments 34A (FIG. 4) and medial segment 34B (FIG. 4) connecting end segments 34A. First and second commissural regions 46, 48 join end segments 34A of first leaflet 16 to end segments 34A of the second leaflet. Medial segments 34B define valve orifice 20 and are configured to move relative to each other from a separated state (FIG. 5) to a coaptated (or sealed) state (FIG. 4) and back to the separated state.

In some embodiments, each of end segments 34A is sized from about 1 mm to about 2 mm. The size of end segments 34A can be selected based on the size of entire top edge 34. For example, the size of end segments 34A can be less than two percentage points or less than one percentage point of the size of entire top edge 34.

End segments 34 sized as described above have been found by applicant to result in good valvular function.

In some embodiments, other sizes for end segments 34 are used.

In some embodiments, flexible tube 12 (FIG. 1) is configured to maintain an internal circumference without substantial enlargement upon application of an expansion force to flexible tube 12. Internal diameter 58 of does not substantially enlarge in response to radially outward forces 60 that might be experienced by flexible tube 12 after implantation in a patient. Internal diameter 58 refers to the maximum radial dimension passing through the center of flexible tube 12 when flexible tube 12 is in a substantially circular state, as shown in the perspective view of FIG. 1. Resistance to substantial enlargement of the internal circumference and internal diameter of flexible tube 12 helps protect flexible valve structure 14 from potential damage and helps maintain good valvular function.

Although flexible tube 12 resists substantial enlargement of its internal circumference and internal diameter 58, flexible tube 12 remains capable of bending and conforming, as least in part, to surrounding biological tissue during and after implantation in a patient.

In some embodiments, flexible tube 12 is configured to stretch axially in the direction of arrow 62 (FIG. 1) while still being resistant to substantial enlargement of its internal circumference and internal diameter 58.

In some embodiments, first leaflet 16 and second leaflet 18 are formed of a single, continuous sheet material that is neither human tissue nor animal tissue. The sheet material can be a man-made, synthetic material, such as polytetrafluoroethylene (PTFE). PTFE is a synthetic fluoropolymer of tetrafluoroethylene. A PTFE sheet having a thickness of 0.1 mm can be used to form first leaflet 16 and second leaflet 18. Use of a PTFE sheet can make first leaflet 16 and second leaflet 18 substantially fluid tight.

In addition to PTFE, other types of synthetic polymers suitable for implantation can be used.

In some embodiments, flexible tube 12 is formed of a material that is neither human tissue nor animal tissue.

In some embodiments, flexible tube 12 is formed of PTFE.

In some embodiments, flexible tube 12 has inner surface 64 (FIG. 1) having a texture that allows for ingrowth or adhesion of biological tissue to inner surface 64 when implanted in a human or animal body.

In some embodiments, flexible tube 12 has outer surface 66 (FIG. 1) that that is substantially fluid-tight.

In some embodiments, outer surface 66 is substantially smooth and resistant to tissue ingrowth in comparison to inner surface.

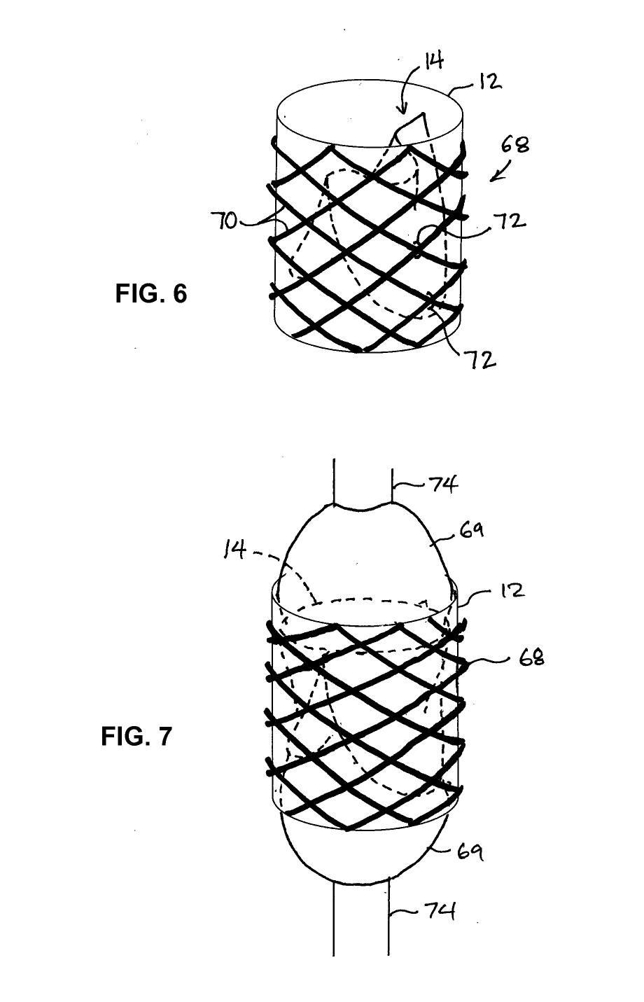

As shown in FIG. 6, in some embodiments tubular stent 68 is used to carry flexible tube 12 and flexible valve structure 14. Flexible tube 12 is disposed within tubular stent 68. Tubular stent 68 comprises interconnected stent struts 70.

In some embodiments, struts 70 are formed of thin metal wires.

In some embodiments, sutures 72 connect flexible tube 12 to tubular stent 68. Only a few sutures 72 are illustrated for ease of illustration. It will be understood that additional sutures are used to secure multiple areas of flexible tube 12 to tubular stent 68.

As shown in FIG. 7, in some embodiments catheter 74 is used to temporarily carry flexible tube 12 and flexible valve structure 14. Catheter 74 extends through flexible tube 12 and valve orifice 20. Catheter 74 is removably engaged to flexible tube 12 and flexible valve structure 14.

In some embodiments, tubular stent 68, flexible tube 12, and flexible valve structure 14 are crimped and/or collapsed onto catheter 74 to allow for delivery through an anatomical lumen like a blood vessel. For example, catheter 74 can be used to pass flexible tube 12 and flexible valve structure 14 through a patient's vasculature and to position flexible tube 12 and flexible valve structure 14 at a desired treatment site within the patient.

In some embodiments, tubular stent 68 is balloon-expandable and is configured to be crimped or collapsed state during delivery through an anatomical lumen. At the treatment site, balloon 69 of the catheter can be used to forcibly expand tubular stent 68 and deploy flexible tube 12 and flexible valve structure 14 to a functional configuration at the treatment site. After expansion of tubular stent 68, balloon 69 is deflated, and tubular stent 68 maintains its expanded state and engages the surrounding tissue to keep flexible tube 12 and flexible valve structure 14 at the treatment site.

In some embodiments, tubular stent 68 is self-expanding and is configured to spring from a crimped or collapsed state to an expanded state upon removal of a cover sheath or other retaining device on tubular stent 68. At the treatment site, the cover sheath or retaining device is removed to allow tubular stent 68 to self-expand and deploy flexible tube 12 and flexible valve structure 14 to a functional configuration at the treatment site. After expansion, optional protrusions or other features on tubular stent 68 can engage surrounding tissue to keep flexible tube 12 and flexible valve structure 14 at the treatment site.

After deployment of flexible tube 12 and flexible valve structure 14 at the treatment site, the catheter is withdrawn from the patient. After withdrawal, tubular stent 68, flexible tube 12, and flexible valve structure 14 remain at the treatment site.

Embodiments of the present invention include a method of making an implantable valve system.

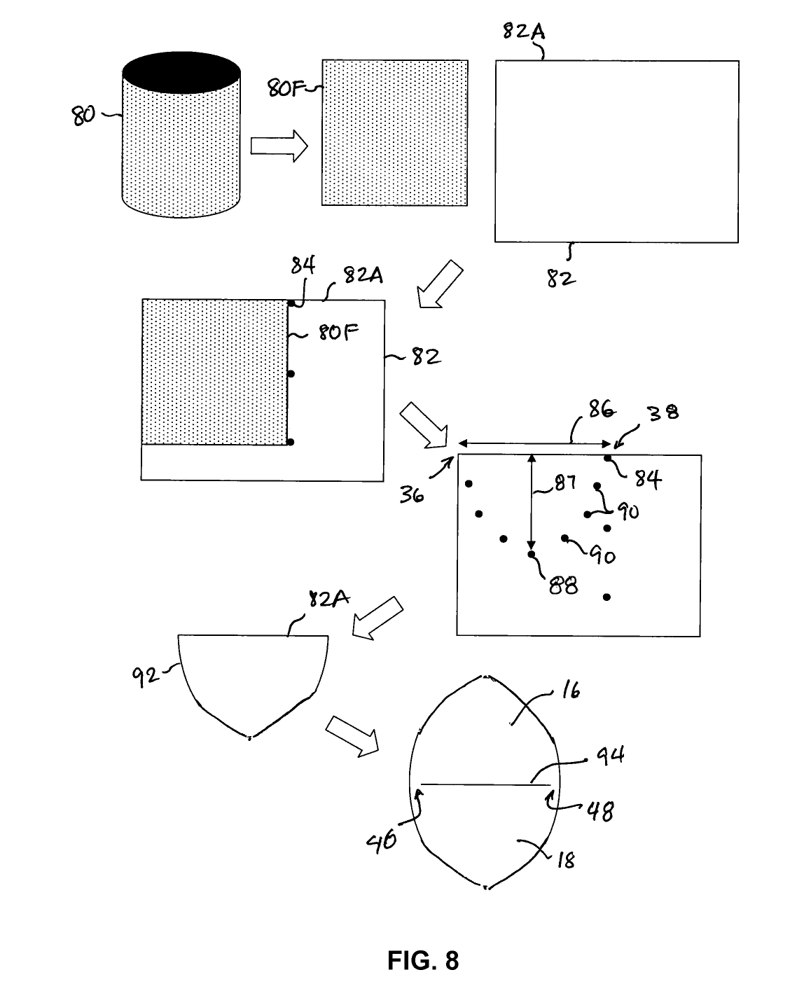

As shown in FIG. 8, an exemplary method comprises compressing a flexible tube material 80 to a flattened state 80F, placing the flattened tube material 80F on a folded sheet material 82, and putting a marking 84 on the folded sheet material at one or more edges of flattened tube material 80F. The flexible tube material 80 corresponds to flexible tube 12. The folded sheet material 82 will become leaflets 16, 18. Fold 82A corresponds to top edges 34 of leaflets 16, 18. Marking 84 indicates the size of top edges 34 or width 86 of leaflets 16, 18.

Next, height 87 of leaflets 16, 18 is determined by placing another marking 88 below the center of width 86. Marking 88 corresponds to sharp point 44 at the apex of lower portions 24 of each leaflet 16, 18. The location of marking 88 corresponds to height 87 being about 10% less than width 86.

Next, cutting of the folded sheet material 82 is performed along substantially parabolic paths from end points 36, 38 of width 86 to marking 88.

Optionally, additional markings 90 are placed between end points 36, 38 and marking 88 prior to cutting. Additional markings 90 provide guide points during cutting.

Cutting along the substantially parabolic path creates a folded sheet 92 having the shape of a bishop mitre. Next, folded sheet is unfolded and straight cut 94 is made at the former location of the fold. Ends of cut 94 are about 1 mm to about 2 mm from the edge of sheet. The material between ends of cut 94 and the edge of the sheet correspond to commissural regions connecting first leaflet 16 and second leaflet 18. Thereafter, first leaflet 16 and second leaflet 18 are sutured to the inner surface of flexible tube material 80.

Prior to suturing, everting of flexible tube material 80 can be performed so that its inner surface faces outward. With inner surface facing outward, suturing of first leaflet 16 and second leaflet 18 is facilitated. Suturing can including placing attachment sutures 50, 52 (FIG. 1) and sutures 32 along the substantially edges of first leaflet 16 and second leaflet 18.

After completion of suturing, flexible tube material 80 is returned to its original configuration with its inner surface facing inward. This places leaflets 16 and 18 within the lumen of flexible tube material 80.

In the above description of various sutures, the sutures can be replaced by other means of attachment, such as fusing, welding, staples, clips, and adhesive bonding, can be used.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

What is claimed is:

1. An implantable valve system comprising:
a flexible tube; and
a flexible valve structure within the flexible tube, the flexible valve structure comprising a first leaflet, a second leaflet, and a valve orifice between the first leaflet and the second leaflet, each of the first leaflet and the second leaflet comprising an upper portion forming the valve orifice and a lower portion attached to the flexible tube, the valve orifice having a maximum circumference less than an internal circumference of the flexible tube.

2. The system of claim 1, wherein each of the lower portions comprises a curved attachment region attached to the flexible tube.

3. The system of claim 2, wherein curved attachment region corresponds to a substantially parabolic edge of the lower portion.

4. The system of claim 2, further comprising sutures connecting the flexible tube to the curved attachment region of each lower portion.

5. The system of claim 1, wherein each of the upper portions includes a top edge sized at about half of the internal circumference of the flexible tube.

6. The system of claim 1, wherein the upper portion of the first leaflet is joined to the upper portion of the second leaflet at a first commissural region and at a second commissural region, and the valve orifice is bounded by the first commissural region and the second commissural region.

7. The system of claim 6, further comprising a first attachment suture at the first commissural region and a second attachment suture at the second commissural region, the first attachment suture connecting the first commissural region to the flexible tube, the second attachment suture connecting the second commissural region to the flexible tube.

8. The system of claim 6, further comprising a first commissural suture at the first commissural region and a second commissural suture at the second commissural region, the first commissural suture located at or about an end of the valve orifice, and the second commissural suture located at or about another end of the valve orifice.

9. The system of claim 6, wherein each of the upper portions includes a top edge, each top edge comprises end segments and a medial segment connecting the end segments, the first and second commissural regions join the end segments of the first leaflet to the end segments of the second leaflet, and the medial segments define the valve orifice and are configured to move relative to each other from a separated state to a coaptated state.

10. The system of claim 9, wherein each of the end segments is sized from about 1 mm to about 2 mm.

11. The system of claim 10, wherein the top edge is sized at about half of the internal circumference of the flexible tube.

12. The system of claim 1, wherein the flexible tube is configured to maintain an internal circumference without substantial enlargement upon application of an expansion force to the flexible tube.

13. The system of claim 12, wherein the flexible tube is configured to stretch axially.

14. The system of claim 1, wherein the first leaflet and the second leaflet are formed of a single, continuous sheet material that is neither human tissue nor animal tissue.

15. The system of claim 1, wherein the flexible tube is formed of a material that is neither human tissue nor animal tissue.

16. The system of claim 1, wherein the flexible tube, the first leaflet, and the second leaflet are formed of PTFE.

17. The system of claim 1, wherein the flexible tube has an inner surface having a texture that allows for ingrowth or adhesion of biological tissue to the inner surface when implanted in a human or animal body.

18. The system of claim 17, wherein the flexible tube has an outer surface that that is substantially fluid-tight.

19. The system of claim 17, wherein the flexible tube has an outer surface that is substantially smooth and resistant to tissue ingrowth in comparison to the inner surface.

20. The system of claim 1, further comprising a tubular stent, wherein the flexible tube is disposed within the tubular stent, and the tubular stent comprises interconnected stent struts.

21. The system of claim 20, further comprising a catheter carrying the flexible tube and the flexible valve, the catheter extending through the flexible tube and the valve orifice.
